(19) United States
(12) Patent Application Publication
(10) Pub. No.: US 2009/0092542 A1
(43) Pub. Date: Apr. 9, 2009

(54) Bioinert tissue marker

(76) Inventor: Stuart K.J. Smyth, Shelburne, VT (US)

Correspondence Address: STUART K.J. SMYTH, P.O. BOX 1059, Shelburne, VT, 05482, US

(21) Appl. No.: 12/220,322

(22) Filed: Jul. 24, 2008

Related U.S. Application Data
(60) Provisional application No. 60/997,951, filed on Oct. 9, 2007.

(51) Int. Cl. A61K 51/00

(52) U.S. Cl. 424/1.29

(57) ABSTRACT

This invention relates to a safe, effective, bioinert microporous radiopaque microparticles or spheres as a method and means to mark or provide “localization” of various tissues within the body and to reduce the incidence of adverse tissue reaction. Further, these same microporous particles or spheres comprising the tissue marker(s) are nonmigratory, nonantigenic, and capable of providing selective therapeutic effects, and whereby the macro-colloidal surfaces of both the macroarchitecture and microarchitecture of the spheres or particles scientifically demonstrate the capacity to absorb toxic products of bacterial decay as well as provide neutralizing electron donation activity at the site of the biopsy or therapeutic intervention as well as proximate to the site of placement; both at the macro-scale of each sphere as well as within the microarchitecture of each particle or sphere, and a bioinert tissue marker whereby the pores on the surface of each sphere are connected to the pores each sphere by blow-holes, and the internal pores are in turn interconnected thus capable of hosting gases, liquids, organic substances, inorganic substances, nanostructures, nanoparticles and nanomaterials.

Reference to US Provisional Patent: This non-provisional patent application is related to prior application U.S. Provisional Patent No. 60/997,951 and cross-referenced to US Patent Application No. 20070128244 published Jun. 7, 2007.

### INTRODUCTION

This invention relates to methods of tissue marking utilizing bioinert microporous ceramic microspheres containing within the porous microarchitecture of each sphere or particle a contrast medium or other pharmaceutical preparations. Preferred microparticles include a permanently radiopaque bioinert microporous ceramic particle or sphere, which may be smooth and/or polished on the macro-scale, porous and uneven on the micro-scale, and having interconnected pores. Pores on the surface of each sphere may be connected to pores inside each sphere via blow-holes and the internal pores are interconnected, so that in addition to a high porosity, the spheres have a high permeability to gas and liquid or substances such as contrast medium or other pharmaceutical preparations. Due to the specific bioinert ceramic macro-colloidal surfaces present at the macro-scale and micro-scale each sphere, the tissue marker may provide selective therapeutic effects and will serve to further absorb toxic products of bacterial decay as well as provide neutralizing electron donation activity at the site of placement.

### BACKGROUND OF THE INVENTION

Tissue marking is a method of marking a position in the body, such as a specific position or organ, to permit the doctor to revisit the initial position at a later time to check progress or developments of an ailment or treatment, or to allow for the re-treatment at the same time. For example, tissue marking can be used during biopsy or other tissue removal procedures to accurately mark the site of tissue removal or biopsy, to allow a doctor to return to the same site if desired so as, for example, to monitor the status of the tissue in question, or to perform another biopsy.

Such tissue marking can be useful in procedures relating to prostate biopsies, breast biopsies, or in procedures relating to rectum or colon biopsies or tissue removal.

During a breast biopsy, it is not uncommon for all evidence of a lesion to be removed from the site, and it makes it difficult for the doctor to return to the same location later, to re-check the site. This problem, created by the removal of a potentially malignant breast mass or cluster of microcalcifications during core biopsy can be ameliorated by placing radiographically visible tissue markers immediately following biopsy.

The tissue marker which is made of a radiopaque material, can be used to help locate the biopsy site in case of malignancy is determined, thereby enabling return to the same site and optionally a subsequent treatment such as surgical excision, even id mammographic findings associated with the original lesion were removed completely.

Another example of an application for tissue marking is in prostate biopsies. Tissue marking can ensure that the tissue of non-determined initial biopsies can be monitored for progressive disease, and if necessary a follow-up biopsy can be performed at the site of the initial biopsy.

Other methods of tissue marking or “localization” are described in medical journal article; “Placement of Endovascular Embolization Microcoils to Localize the Site of Breast Lesions Removal at Sterotactic Core Biopsy.” Radiology, 1998, 206: 275-278 by Fajardo, Bird, Herman and Da Angalis.

Still another method of localizing breast lesions is described in medical journal article, “Preoperative Localization of Non-Palpable Breast Lesions Using Wire Marker and Perforated Mammographic Grid.” Radiology 146: 833-835 by Goldberg, Hall and Simon; see also U.S. Pat. Nos. 4,341,220 and 5,665,092.

### OBJECT OF THE INVENTION

It is therefore an object of the present invention to provide a safe, effective, bioinert microporous radiopaque microparticles or spheres as a method and means to mark or provide “localization” of various tissues within the body and to reduce the incidence of adverse tissue reaction. Further, for these same microporous particles or spheres comprising the tissue marker(s), for the particles or spheres to be nonmigratory, nonantigenic, capable of providing selective therapeutic effects, and whereby the macro-colloidal surfaces of both the macroarchitecture and microarchitecture of the spheres or particles scientifically demonstrate the capacity to absorb toxic products of bacterial decay as well as provide neutralizing electron donation activity at and around the site of placement, both at the macro-scale of each sphere and within the microarchitecture of each particle or sphere wherein, altogether, the pores on the surface of each sphere are connected to the pores inside each sphere by blow-holes and the internal pores are in turn, interconnected.

### SUMMARY OF THE INVENTION

According to the first aspect of the present invention there may be a bioinert microporous ceramic tissue marker comprised of particulate microporous bioinert ceramic material, having interconnected pores.

Further according to the invention the particles, which comprise the tissue marker, may be in the form of different shapes or spheres.

Further, according to the present invention, the spheres may be radiopaque and thus not loose their radiopacity, which may mean that the point of placement of the tissue marker and its surrounding tissue may be monitored on a permanent basis.

Yet further according to the invention, the pores on the surface of each sphere may be connected to at least some of the pores inside each sphere via blow-holes and at least some of these internal pores may be interconnected, so that in addition to high porosity, the spheres have a high permeability to gas, liquid, organic or inorganic substances which may include such things as contrast agent and/or other pharmaceutical preparations.

The sphere(s), which acts as the tissue marker, may have an internal porosity of 30% to 80% per volume.

The invention containing bioinert microporous ceramic spheres having interconnected pores via blow-holes, and which may have a porosity of 30% to 85% by volume, may permit the possibility of increasing the contrast enhancing agent and/or other organic or inorganic substances such as pharmaceutical preparations or chemoattractors, so that each sphere may include a permanent, enhanced radiopaque particle substrate greater than 50% of the spheres mass.

The pores of the particles or spheres may have diameters in the range of 0.3 to 10 micrometers but may be more or less according to the specific tissue marker or “localization” requirements.

The spheres may have a diameter larger than 45 micrometer, range from 85 to 1000 micrometers, more preferably 85 to 500 micrometers.

The bioinert ceramic material microporous tissue marker may be injectable through a hypodermic needle of varying gauge size or cannula mechanism.

The preferred bioinert ceramic material utilized to create the microporous tissue marker according to this invention may be comprised of sintered aluminum oxide.

The bioinert ceramic material utilized to create the microporous tissue marker may be comprised of microporous aluminum oxide (alumina) that may be macro-colloidal surfaces, both at the macro-scale and within the micro-scale within each sphere, which may demonstrate the capability of absorbing toxic products of bacterial decay but may not possess any actual antibacterial activity upon exposure to liquids.

Further according to the invention, the bioinert microporous spheres of sintered aluminum oxide (alumina) may have scientifically demonstrated the capability of providing neutron electron donation activity at and around the site of placement of the tissue marker.

Further, according to the invention, the bioinert microporous spheres comprised of sintered aluminum oxide (alumina) may be combined and/or coated with other bioinert and/or biocompatible ceramic based materials such as microporous zirconium oxide, microporous hydroxyapatite, calcium phosphates and tricalcium phosphates; all according to the particular type of tissue and/or tissue application as specified by the caregiver.

Further according to the invention the spheres may be able to absorb, retain, distribute or withstand temperature fluctuations, high heat, varying light intensity, radiation, varying electromagnetic fields, chemicals and those therapeutic applications which may be applied at the tissue marking site and within the surrounding local host tissue.

Yet further to the invention, one or more bioinert microporous ceramic spheres may be placed at the point of origin of the biopsy or treatment site.

Yet further to the invention, one or more bioinert microporous spheres may be placed around or situated proximate to the site of biopsy or place of treatment.

Yet further to the invention, the bioinert microporous ceramic spheres made of aluminum oxide (alumina) that once implanted, may maintain their original shape and size over an extended period of time, even when coatings and/or other gases, liquids, organic or inorganic materials have been resorbed by the body.

Yet further to the invention of a bioinert microporous ceramic sphere, with a porosity of 30% to 80% per volume may maintain its tissue volume, stability and/or structural tissue integrity beyond the date of the original placement of such a sphere.

The bioinert microporous ceramic spheres that comprise the tissue markers may be prepared, but not limited to; a method including the steps of;

1) Milling the pure precursor ceramic raw material into powder with particle size finer than 1 micrometer

2) Blending a combustible substance known in the trade of manufacturing porous structures into powders

3) Mixing the powder with water to form a paste

4) Forming solid spherical particles at a temperature of 1350 C to 1550 C to form inert microporous ceramic spheres having pore sizes of between 0.3 to 10 micrometers at a diameter of between 85 to 100 micrometers, and, screening the spheres into preselected, preferably but not exclusively, narrow size fractions, for example 45 to 110 micrometer, 100 to 200 micrometer, 200 to 300 micrometer and so forth.

The applicants have found according to the present invention that bioinert microporous, radiopaque ceramic spheres containing interconnected pores via blow-holes, and which may absorb toxic products of bacterial decay and demonstrate the capability of providing electron donation activity both at the macro-scale and micro-scale level of each sphere, will meet if not exceeds, most or all of the requirements for a bioinert radiopaque tissue marker.

The descriptions of the embodiments of the present invention as given above may be for understanding the present invention. It will be appreciated further that the invention may not be limited to the particular embodiments described herein, but is capable of various modifications, rearrangements and substitutes as will be apparent to those skilled in the art without departing from the scope of the disclosure.

1) A bioinert microporous ceramic tissue marker comprised of particulate microporous bioinert ceramic material, having a highly porous microarchitecture inside the tissue marker(s) and the internal pores within the microarchitecture of each sphere are interconnected.

2) The particles, which according to claim 1, that comprise the tissue marker, will be in the form of different spheres, shapes, design, architecture, varying sizes, and may also be contained in a composite or matrices.

3) A bioinert microporous bioceramic tissue marker, which according to claims 1-2 are comprised of spheres an the spheres will be radiopaque thereby not loosing their radiopacity, meaning that the point of placement of the tissue marker and its surrounding tissue may be monitored on a permanent basis or for the lifetime of the patient who has received the tissue marker(s).

4) A bioinert microporous bioceramic tissue marker, according to claims 1-3, with pores on the surface of each sphere connected to at least some of the pores inside each sphere via blow-holes and at least some of these internal pores will be interconnected, so that in addition to high porosity, the spheres have a high permeability to gas, liquid, organic or inorganic substances which may include, but not limited to, such things as contrast agent and/or other pharmaceutical preparations.

5) A bioinert microporous bioceramic tissue marker(s), according to claims 1-4, whereby the porous microarchitecture will permit gases, liquids, organic and inorganic substances to be contained or coated either temporarily or permanently within the microarchitecture of each sphere or particle. Such substances may include but are not limited to nanosubstances, nanomaterials and nanostructures, polymers and polymer coatings, copolymers and copolymer coatings, hydrogels and gel based coatings, metallic substances and metallic coatings, nanoparticles and metallic nanoparticles, minerals and mineral based coatings, corundum based minerals and corundum based coatings, diamonds and diamond coatings, myoblasts, fibroblasts, tokeratin, silk, fibrin, collagen, gelatin, alginic acid and salts, chitin, chitosan, hyaluron, hyaluronic acid, cellulose, n-acetyl glucosamine, proteoglycans, glycolic acid polymers, lactic acid polymers, glycolic acid/lactic acid co-polymers, fibrin cross-linker, calcium ions, sucrose, lactose, maltose, dextrose, mannose, trehalose, sorbitol, albumin, sorbate, polysorbate, sodium bicarbonate/citric acid, sodium bicarbonate/acetic acid, calcium carbonate/acetic acid, antibiotics, anticoagulants, steroids, various drugs, growth factors, antibodies (poly and mono), chemoattractors, anesthetics, a ntiproliferatives/antitumor agents, a ntivirals, vaccines, cytokines, colony stimulating factors, antifungals, antiparasitics, antiinflammatories, peptides, antiseptics, hormones, vitamins, glycoproteins, fibronectin, peptides, proteins, carbohydrates, proteoglycans, antigens, nucleotides, lipids, liposomes, fibrinolysis inhibitors and gene therapy reagents.

6) A bioinert microporous ceramic spheres, according to claims 1-5, whereby the sphere(s), which are the tissue marker(s), will have an internal porosity of 30% to 80% per volume.

7) A bioinert microporous ceramic sphere(s), according to claims 1-6, having interconnected pores via blow-holes, and which may have a porosity of 30% to 80% by volume, will permit for the possibility of increasing the contrast enhancing agent and/or other organic or inorganic substances such as pharmaceutical preparations or chemoattractors, so that each sphere may include a permanent, enhanced radiopaque particle substrate greater than 50% of the spheres mass.

8) A bioinert microporous bioceramic tissue marker, according to claims 1-7, wherein the pores of the particles or spheres will have diameters in the range of 0.3 to 10 micrometers, but may be more or less according to the specific tissue marker or “localization” requirements.

9) A bioinert microporous bioceramic tissue marker, according to claims 1-8, whereby the spheres may have a diameter larger than 45 micrometer, range from 85 to 1000 micrometers, more preferably 85 to 500 micrometers; and when combined to include more than one sphere will form a composite which may contain spheres of varying sizes.

10) The bioinert ceramic material microporous tissue marker, according to claims 1-9 can be injected through a hypodermic needle of varying gauge size or other cannula or introductory mechanism.

11) A bioinert microporous bioceramic tissue marker, according to claims 1-10, where the preferred bioinert ceramic material utilized to create the microporous tissue marker according to this invention will be comprised of sintered aluminum oxide with macro-colloidal surfaces and macro-colloidal surfaces present with both the macroarchitecture and microarchitecture of the spheres.

12) The bioinert microporous ceramic material, according to claims 1-11, utilized to create the microporous tissue marker that will be comprised of microporous aluminum oxide (alumina) that will have macro-colloidal surfaces, both at the macro-scale and within the micro-scale within each sphere, and which demonstrate the capability of absorbing toxic products of bacterial decay but do not possess any actual antibacterial activity upon exposure to liquids.

13) The bioinert microporous bioceramic tissue markers, according to claims 1-12, where the spheres consisting primarily of sintered aluminum oxide (alumina) have scientifically demonstrated the capability of providing neutron electron donation activity at and proximate to the site of placement of the tissue marker(s).

14) The bioinert microporous bioceramic tissue marker(s), according to claims 1-13, whereby the spheres are comprised of sintered aluminum oxide (alumina) and may be combined and/or coated with other bioinert and/or biocompatible ceramic based materials such as microporous zirconium oxide, zirconium oxide, microporous hydroxyapatite, hydroxyapatite, calcium phosphates and tricalcium phosphate; and all materials selected for the particular type of tissue and/or tissue application/treatment as specified by the caregiver.

15) A bioinert microporous bioceramic tissue marker(s),according to claims 1-14, whereby the spheres are multi-capable and will absorb, retain, process, distribute, redistribute; various therapeutic energies, withstand temperature fluctuations such as high or elevated heat, work in conjunction with targeted radiowave therapy, capable of absorbing or deflecting varying light based therapies and intensities, become radioactive, gradually lose their radioactivity, working with and within varying electromagnetic fields, are ultrasonically compatible and capable of becoming ultrasonically enabled, and which may be used with varying chemicals and those therapeutic applications which may be applied at the tissue marking site and within the surrounding local host tissue.

16) A bioinert microporous bioceramic tissue marker, according to claims 1-15, whereby one or more or a multiple number of bioinert microporous ceramic spheres will be placed at the point of origin of the biopsy or treatment site.

17) A bioinert microporous bioceramic tissue marker(s), according to claims 1-16, whereby one or more or a multiple number of bioinert microporous spheres may be placed around or situated proximate to the site of biopsy or place of treatment and which will further serve to extend the function of macro-colloidal surfaces which absorb toxic products of bacterial decay and provide neutron electron donation activity at and proximate to the site biopsy, treatment or of the actual placement of the tissue marker.

18) A bioinert microporous bioceramic sphere(s), according to claims 1-17, which are made of aluminum oxide (alumina) and that once implanted, will maintain their original shape and size permanently, even when coatings and/or other gases, liquids, organic or inorganic materials, nanostructures, nanoparticles or other nanomaterials have been resorbed by the body or when the coatings and additive substances have exhausted their potential or have been fully utilized for a therapeutic or ongoing therapeutic treatment(s).

19) A bioinert microporous bioceramic sphere(s), according to claims 1-18, and with a porosity of 30% to 80% per volume and the microporous interconnected microarchitecture will aid in maintaining sustained tissue volume following biopsy or therapeutic intervention, maintain tissue stability and/or structural tissue integrity beyond the date of the original placement of such a sphere(s) or following biopsy or other therapeutic intervention(s).

20) A bioinert microporous bioceramic sphere, according to claims 1-19, with a porosity of 30% to 85% containing a porous microarchitecture with interconnected pores that creates an environment for maximum healthy tissue ingrowth thereby preventing migration of the spheres or tissue marker to unintended or unnecessary places within the body.
